(19) United States
(12) Patent Application Publication
(10) Pub. No.: US 2011/0282256 A1
(43) Pub. Date: Nov. 17, 2011

(54) ORTHOPEDIC SHOULDER SYSTEM AND METHOD

(75) Inventor: Michael F. Carroll, Traverse City, MI (US)

(73) Assignee: JOINT SMART LLC, Traverse City, MI (US)

(21) Appl. No.: 13/190,945

(22) Filed: Jul. 26, 2011

Related U.S. Application Data
(63) Continuation-in-part of application No. PCT/US2010/021993, filed on Jan. 25, 2010.
(60) Provisional application No. 61/147,241, filed on Jan. 26, 2009.
(60) Provisional application No. 61/147,171, filed on Jan. 26, 2009.

(51) Int. Cl. A61F 5/00

(52) U.S. Cl. 602/20

(57) ABSTRACT

A system comprising a combination of orthopedic traction devices for limiting abduction of the arm and preventing impingement of the user's shoulder. The devices include a nighttime ipsilateral shoulder traction device with removable leg and wrist cuffs, an elastic connecting strap connecting the cuffs to apply impingement-reducing traction to the shoulder joint; and a daytime centrifugal exercise device with a weighted member rotated by a handle using the ipsilateral arm. The invention also includes a method for using the two devices in combination, and a series of exercises for the centrifugal exercise device that specifically complement the nighttime impingement reduction of the traction device. In the preferred form, the traction device includes removable leg and wrist cuffs secured to the lower leg and wrist, respectively, and a one-handed quick release mechanism that allows the user to release the cuffed arm with the cuffed hand.

### RELATED APPLICATIONS

This application is a continuation in part of PCT patent application PCT/US2010/021993, filed 25 Jan. 2010, which is incorporated herein by reference, and which claims the benefit of U.S. Provisional Application Nos. 61/147,241 and 61/147,171, both filed Jan. 26, 2009 by the same inventor, the entirety of which provisional applications is incorporated herein by reference.

### FIELD OF THE INVENTION

The invention is in the field of orthopedic treatment devices for preventing impingement in a patient's shoulder, particularly for treating rotator cuff tendonitis.

### BACKGROUND OF THE INVENTION AND DESCRIPTION OF RELATED ART

The shoulder enjoys a 270-degree range of motion, the greatest range in the human body. This freedom requires a remarkably complex muscle and joint interaction. This task is largely done by the four muscles that make up the rotator cuff. These muscles “cuff” the ball of the humerus and keep the ball within the socket of the glenoid fossa. When this process is corrupted, one muscle of the rotator cuff is impinged between two hard surfaces. This process then leads to degeneration and further disease of the supraspinatus tendon and subsequently the rotator cuff complex. Thus, reduction of movement and forces that cause impingement on the supraspinatus tendon is assumed to reduce its degeneration. Maximum impingement occurs when a person's arm is in abduction, or away from the body. Abduction in this manner frequently occurs in circumstances when the body is sleeping. Today's sleeping surfaces, with firm support maintaining the body in a relatively flat position, produce circumstances where a person's arms are frequently in an abducted position.

Impingement is less a problem when the body is upright, because the inherent weight of a person's arm provides necessary and constant traction to keep the head of the humerus from impinging the supraspinatus tendon against the coracoacromial arch. (Another name for “rotator cuff tendonitis” is “impingement syndrome”.) During the nighttime, in normal sleep or otherwise when a person is in a supine or prone position, the shoulder loses the built-in traction protection provided by gravity.

My U.S. patent application Ser. No. 11/159,942, published as US 2006/0293623 on Dec. 28, 2006, discloses my prior orthopedic treatment device and method to prevent impingement of a patient's supraspinatus tendon while the patient is sleeping or is otherwise in a supine or prone position. The device limits abduction of the arm by exerting direct traction on the arm in a downward direction. The device consists of a first sleeve or cuff removably fixed around the patient's thigh, and a second sleeve or cuff removably fixed around the patient's wrist, both ipsilateral (on the same side as, or “proximal” in my prior application) to the shoulder treated, with the sleeves connected by a resilient strap that exerts traction on the arm.

While effective in treating rotator cuff tendonitis, some practical disadvantages of my prior device have been discovered, including: inconsistent traction given the typical locations and relative movement of the wrist and thigh while sleeping; difficulty or awkwardness in removing the device; a perceived feeling of discomfort or anxiety at having an arm partly immobilized during the night when use of the arm being treated might become necessary; and imperfect fitting due to anatomic variability at the level of the human thigh.

Also, the foregoing orthopedic traction device only helps prevent impingement while sleeping, through traction that maintains a simulated free-hanging arm position. However, while awake a person is frequently called upon to put his arm in abduction, causing impingement throughout the day. While it is generally recognized that strengthening the muscles that surround and stabilize the shoulder can help prevent impingement when actively using the arm and shoulder in abduction, shoulder exercises to date have not been directed toward specifically strengthening those muscle groups that reinforce and complement the nighttime impingement-preventing traction of an orthopedic arm-restraining device.

### BRIEF SUMMARY OF THE INVENTION

According to the invention, an orthopedic kit to prevent impingement in a user's shoulder comprises a traction device for preventing abduction of the user's ipsilateral (same side) arm while the user is in a sleeping position and a centrifugal exercise device for use when the user is awake and upright for strengthening the rotator cuff impingement-preventing muscle group of the shoulder of the user. The traction device comprises a wrist cuff adapted to be removably secured to the user's ipsilateral wrist or lower arm, a leg cuff adapted to be removably secured to the user's ipsilateral (same side) leg; and a strap member connected between the wrist cuff and the leg cuff and of a length to apply impingement-reducing traction on the user's ipsilateral arm and thus on the user's shoulder while in a sleeping position and with the user's ipsilateral arm alongside the user's body. The centrifugal exercise device comprises a handle and a weighted member rotatably connected to the handle through a short tether so that a user gripping the handle with the ipsilateral arm unbent can rotate the weighted member in an essentially circular path that defines a plane substantially perpendicular to the unbent ipsilateral arm of the user. The strap exerts downward traction on the wrist and thus on the shoulder in a position similar to a suspended at-rest arm position as provided by gravity when the user is upright, in order to prevent impingement while sleeping. Preferably, the strap is elastic.

In one embodiment, the leg cuff is adapted to be attached to a thigh portion of the user's ipsilateral leg. Preferably, the leg cuff is adapted to be attached to a below-the-knee portion of the user's ipsilateral leg.

In another embodiment, the strap member includes a quick release mechanism for breaking the strap connection between the wrist cuff and the leg cuff while leaving the wrist and leg cuffs attached to their respective wrist and leg.

In another embodiment, the kit includes multiple centrifugal exercise devices, each with a weighted member of a different weight value. Preferably, the magnitude of the weighted members is less than one pound.

The invention further comprises a method for using the above orthopedic kit to strengthen a user's shoulder with “nighttime” (supine or prone) impingement preventing traction device and the “daytime” (awake and active) centrifugal exercise device in combination, and more particularly a method of using the centrifugal exercise device for one or more exercises selected from the group consisting of a front vertical plane rotation; a side vertical plane rotation; a diagonal vertical plane rotation; and a downward horizontal plane rotation.

According to the invention, the method for using the above orthopedic kit to strengthen a user's shoulder comprises (1) preventing impingement during a nighttime sleeping phase using the traction device for preventing abduction of the user's ipsilateral arm while the user is in a sleeping position and (2) strengthening the user's ipsilateral rotator cuff muscle group of the user's shoulder during an upright phase by using the centrifugal exercise device with the user gripping the handle and rotating the centrifugal exercise device in a circle with the user's ipsilateral arm essentially straight in a manner so that the weighted member defines a plane.

The impingement act can be carried out by (a) removably securing the wrist cuff to the user's ipsilateral wrist or lower arm, (b) removably securing the leg cuff to the user's ipsilateral leg; and (c) tensioning the elastic strap member between the wrist cuff and the leg cuff to apply impingement-reducing traction on the ipsilateral arm and thus on the ipsilateral shoulder.

In one embodiment, the rotating act includes rotating the centrifugal exercise device at a speed sufficient for the weighted member to define at least one of a front vertical plane, a side vertical plane, a diagonal vertical plane, and a downward horizontal plane conducted with the user's ipsilateral arm essentially straight.

The term “elastic strap” includes any resilient, stretchable elongated structure capable of connecting the two cuffs so as to provide traction-inducing tension on the arm and shoulder when the spacing of the wrist from the leg cuff is equal to or greater than their natural spacing when a person is standing upright with the arm hanging down freely toward the leg. The term “tether” includes both elastic and inelastic lengths of cord, cable, strap, chain, and the like, and could even include essentially rigid members for connecting a weight rotatably to a handle.

Further according to the invention, an improved impingement-preventing traction device for a user's arm and shoulder in which a first sleeve or cuff is removably secured to the wrist of the arm/shoulder being treated, and a second sleeve or cuff is removably secured to the calf or lower leg (below the knee) of the user's same-side or “ipsilateral” leg, the two cuffs being connected by an elastic strap that exerts downward traction on the wrist/arm and thus on the shoulder, biasing them to a position similar to a suspended at-rest arm position as provided by gravity when the user is upright, in order to prevent impingement while sleeping (or otherwise supine or prone for any reasonable length of time, hereafter generally referred to as “sleeping”). The term “elastic strap” includes any resilient, stretchable elongated structure capable of connecting the two cuffs so as to provide traction-inducing tension on the arm and shoulder when the spacing of the wrist from the lower leg is equal to or greater than their natural spacing when a person is standing upright with the arm hanging down freely toward the leg. The traction-inducing tension is preferably within the range of 1 lb to 7 lbs (pounds) of constant traction when slack is not present in the strap.

In one embodiment, a wrist-to-lower leg traction device can have a quick-release connection of the wrist cuff to the lower leg cuff, the quick-release connection capable of being operated with the cuffed hand to release the cuffed arm from its tractive connection to the lower leg without having to undo or remove either cuff. The quick-release connection can be located on the strap between the two cuffs, allowing the user to tension the strap with the wrist prior to releasing the connection to improve the speed and ease with which the cuffed hand can be freed with either the ipsilateral (cuffed) or non-ipsilateral (free) hand.

Still further according to the invention, a method for strengthening muscles that surround and stabilize a person's shoulder comprises (1) resiliently connecting the person's ipsilateral arm to the person's ipsilateral leg with traction while the person's ipsilateral arm is alongside the person's body; (2) maintaining the person's ipsilateral arm with the person's ipsilateral arm alongside the person's body during a first time period while the person is in a sleeping position; (3) subsequent to the first time period, disconnecting the person's ipsilateral arm from the ipsilateral leg; and (4) while the person in in an upright position and during a second time period, rotating the person's ipsilateral arm in a straight, unbent condition through small circles while holding with the hand of the ipsilateral arm a handle of a centrifugal device that has a weighted member on the end of a flexible connector between the weighted member and the handle.

Preferably, the speed of rotation of the person's ipsilateral arm is sufficient to rotate the weighted member about a circle that defines a plane that is substantially perpendicular to the person's straight, unbent ipsilateral arm.

In one embodiment, the rotation act can be carried out with the person's ipsilateral arm positioned forwardly of the person's body and substantially horizontal. In another embodiment, the rotation act can be carried out with the person's ipsilateral arm positioned laterally of the person's body and substantially horizontal. In another embodiment, the rotation act can carried out with the person's ipsilateral arm positioned forwardly of the person's body and substantially vertical while the person is bent over at the waist.

Further, the rotating step can be repeated during successive time periods with weights of increasing magnitude. The magnitude of the weighted member can vary depending on the strength of the user's arm. Typically, the magnitude of the weighted member can be increased as the user's arm is strengthened over a period of days or weeks of exercises. The magnitude of the weighted member is typically below 1 pound. It has been found that a satisfactory distribution of weighed members is between 1.5 and 7.5 oz. A set of four centrifugal devices progressive weights of a magnitude of 1.5, 3.5, 5.5 and 7.5 oz. has been found to be a best mode of carrying out the invention.

In still another embodiment, the maintaining act is further carried out with the person's ipsilateral arm in traction at least part of the first time period while the person is in a sleeping position.

These and other features and advantages will become apparent from the detailed description below, in light of the accompanying drawings.

### BRIEF DESCRIPTION OF THE DRAWINGS

FIG. 1 is a perspective view of a portion of a patient's torso and ipsilateral arm and leg (looking at the right side of the patient), showing the preferred embodiment of the inventive traction device attached to the patient's wrist and lower leg, with the direction of tractive force exerted by the leg cuff on the wrist cuff illustrated in the direction of the arrow.

FIG. 2 is a perspective view of the traction device of FIG. 1, with one of the cuffs partially opened.

FIG. 2A is similar to FIG. 2, with a schematic illustration of an optional force-adjusting/measuring device.

FIG. 3 is similar to FIG. 1, but shows the patient operating a quick-release connector on the strap adjacent the wrist cuff using the ipsilateral (cuffed) hand.

FIG. 4 is similar to FIG. 3, but shows an alternate quick-release connector located on or adjacent the leg cuff, also operable by the patient's ipsilateral hand.

FIG. 5 is a perspective view of the traction device of FIGS. 1-4 and of a centrifugal exercise device used in a nighttime/daytime combination with the traction device.

FIG. 6 is a front view of a person using the centrifugal exercise device of FIG. 5 for a front vertical plane rotation exercise.

FIG. 7 is a side view of the person in FIG. 6 using the centrifugal exercise device of FIG. 5 for a side vertical plane rotation exercise.

FIG. 8 is a plan view showing the person in FIG. 6 using the centrifugal exercise device of FIG. 5 for a diagonal vertical plane rotation exercise (solid lines), and also showing the front and side vertical plane rotations of FIGS. 6 and 7 (phantom lines).

FIG. 9 is a front view of the person of FIG. 6 using the centrifugal exercise device of FIG. 5 for a downward horizontal plane rotation exercise.

### DETAILED DESCRIPTION OF THE INVENTION

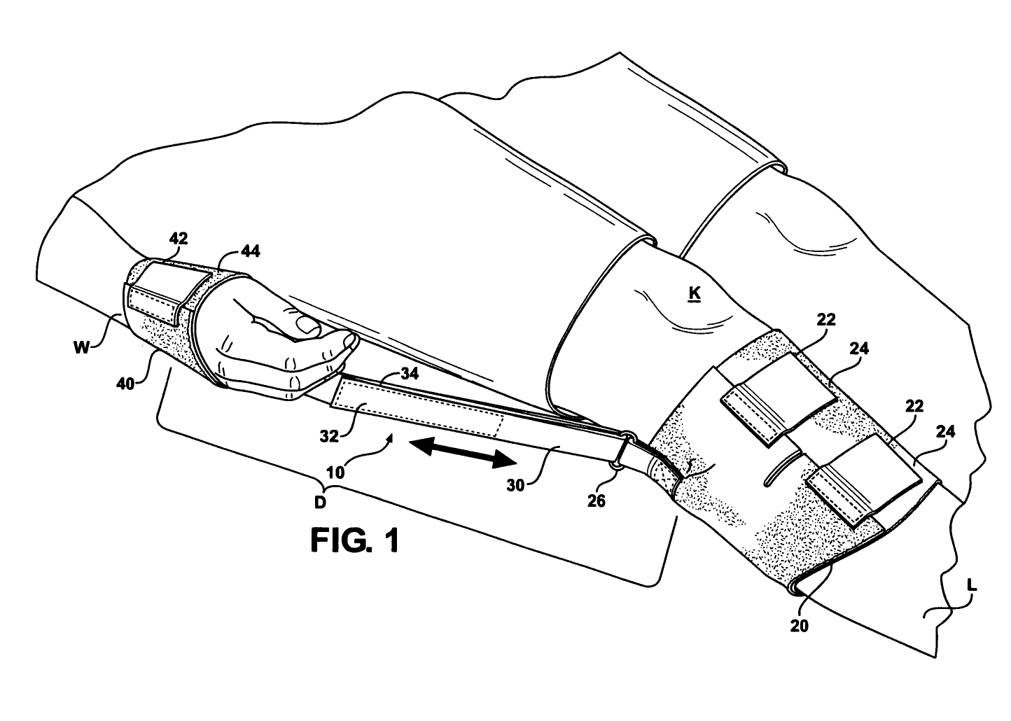

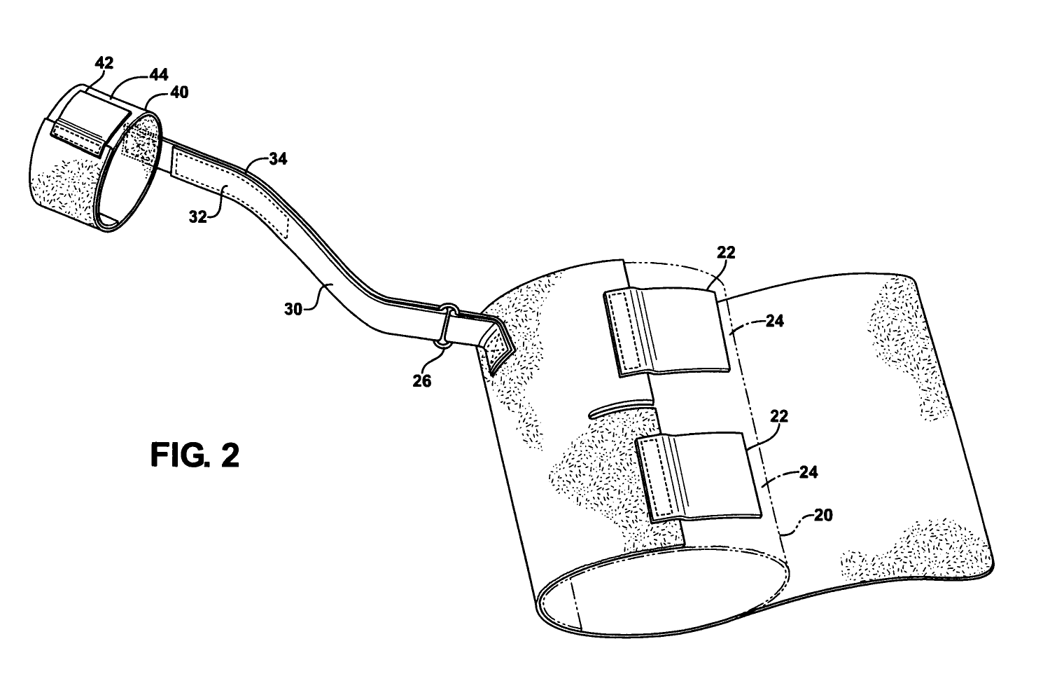

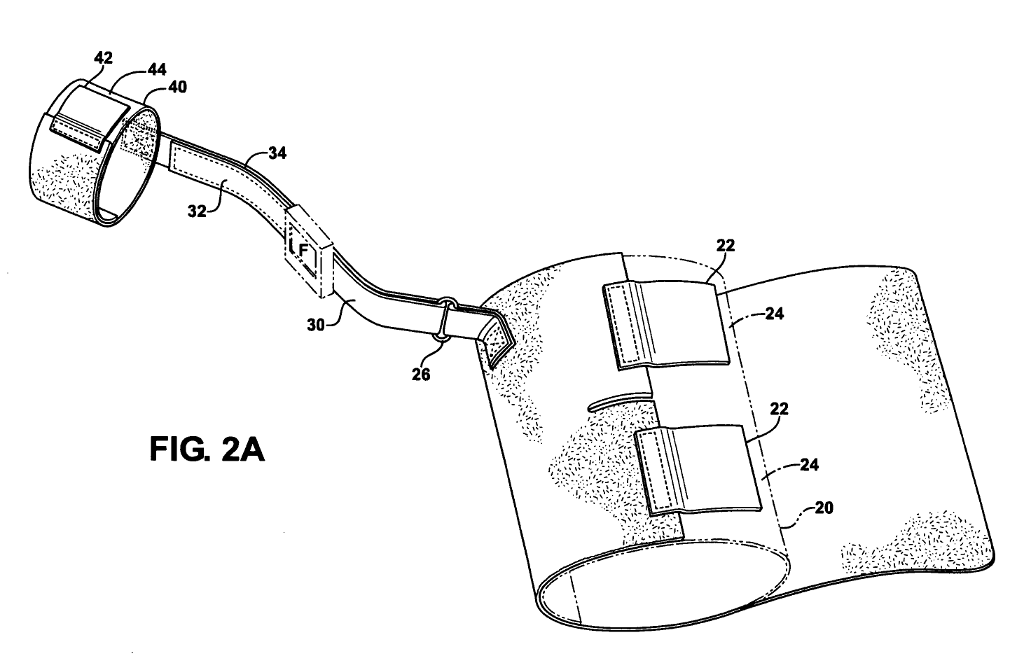

Referring first to FIGS. 1 and 2, the traction device portion of the invention is illustrated in a preferred example at 10 with a wider, larger diameter leg sleeve or cuff 20 connected by an elastic strap 30 to a narrower, smaller diameter wrist sleeve or cuff 40. Cuffs 20 and 40 are made from a soft, conforming, comfortable material such as fabric or soft polymer that can be wrapped around a patient's respective leg L and wrist W securely enough to keep the cuffs in place and to put the strap 30 under tension. In the preferred, illustrated embodiment, cuffs 20 and 40 are rectangular strips or bands of fabric with male hook-and-loop fasteners 22 and 42 on one end that are removably fastened to corresponding fasteners or outer cuff faces 24 and 44 of female hook-and-loop material to form a closed, tubular sleeve or cuff. Of course, other known forms of releasable fastener or connector could be used to secure the free ends of the cuffs to one another to removably secure them around the patient's leg and wrist. And it would be possible, although less convenient, to form cuffs 20 and 40 as simple tubular bands of elastic material designed to be applied by sliding them over the hand and foot, and to fit snugly around the lower leg and wrist, respectively.

While connecting strap 30 is illustrated as a flat band of elastic fabric, it could take other forms of elastic or resilient construction, such as elastic cord or a spring member, provided it exerts a tractive tension on the patient's wrist when connected to and stretched between cuffs 20 and 40 secured on the patient's lower leg and wrist, respectively. For optimal effect when in use, the length of strap 30 in its shortest, relaxed state is preferably no greater than the natural distance D between the user's wrist W and the upper part of the ipsilateral lower leg L, just below the knee, when the user is in a standing position and the arm is suspended by gravity in its normal position. In practice, the length will usually be between about 12 inches and 24 inches. In the preferred form, the shortest, relaxed length of strap 30 will be less than this natural distance D between the wrist and upper part of the lower leg, in order to maintain a constant traction on the wrist and shoulder. Strap 30 can be of fixed length, with its elasticity providing any necessary length adjustment, but can also be provided with a buckle or other adjuster to adjust its length. As shown schematically in FIG. 2A at F, it would be possible to use an in-line tensioning device in an elastic or non-elastic cord or strap, such as a mechanical or electromechanical turnbuckle or adjustable spring member, to supply or adjust the traction-inducing tension in strap 30. It would also be possible to use an in-line force-measuring device in the strap, for example a load cell (transducer) or spring tension gauge in order to measure the amount of traction, either separately or preferably in combination with the in-line tensioning device F. These features would be useful under a physician's prescription to vary, on a nightly basis, the exact amount of tension applied to the shoulder joint by device 10.

As best shown in FIG. 1, cuff 20 is secured to the patient's calf or lower leg L, below the knee. This increases the effective distance between the wrist and leg cuffs 20 and 40 versus a thigh-mounted location, thus increasing the traction-inducing tension exerted through strap 30 on wrist cuff 40 (and thus on the patient's ipsilateral shoulder) when the patient's arm is extended fully downward. Additionally, the below-the-knee attachment of cuff 20 better accommodates variations in patients' relative height and arm and leg lengths, and more securely anchors the leg cuff 20 to the patient's leg, since the protuberance and bend of the knee K functions as a stop to prevent cuff 20 from sliding or being pulled upwards under the tension exerted by the patient's arm and wrist through cuff 40 and strap 30. The knee's anchoring function for cuff 20 is especially important during the typical tossing and turning of the user throughout a night's sleep, and reduces the need to fit cuff 20 in an uncomfortably tight or circulation-restricting manner around the leg in order to prevent slippage.

Another advantage of the lower leg placement of cuff 20 is that it generally allows a single cuff size to fit a larger number of patients' legs. The anatomic variation in lower leg circumference is generally much less than the anatomic variation in thigh circumference, for example with 8″-20″(inches) being a typical range for lower leg circumference among the general population, while 12″-40″ (inches) is a typical range for thigh circumference.

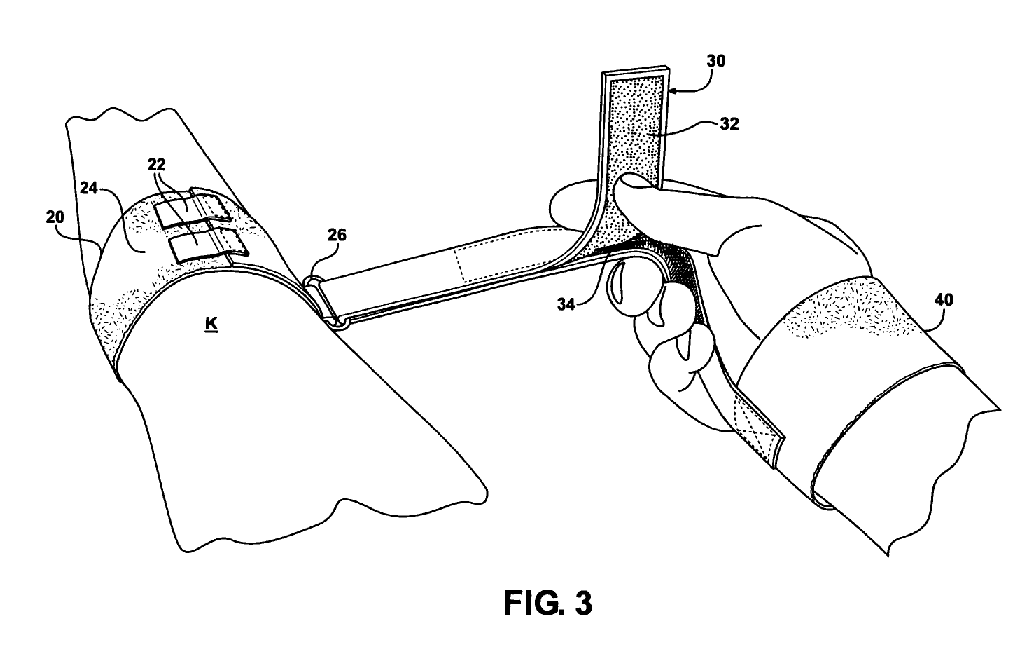

Still referring to FIGS. 1 and 2, and now also to FIG. 3, connecting strap 30 is provided with a release mechanism 32, 34 that allows the wrist cuff 40, still fastened around the patient's wrist, to be disconnected from lower leg cuff 20 fastened around the patient's leg. In FIGS. 1-3 the release mechanism is a strip of hook-and-loop material 32 on a free end of strap 30, material 32 normally being used to attach the free end of strap 30 to another part of the strap, for example a mating strip or patch or outer surface fabric 34 of hook-and-loop material, to form a closed loop passing through a buckle 26 on or secured to leg cuff 20. By pulling the mated hook-and-loop material 32, 34 apart, the closed loop is broken, allowing strap 30 to be pulled out through buckle 26 to free wrist cuff 40 from leg cuff 20.

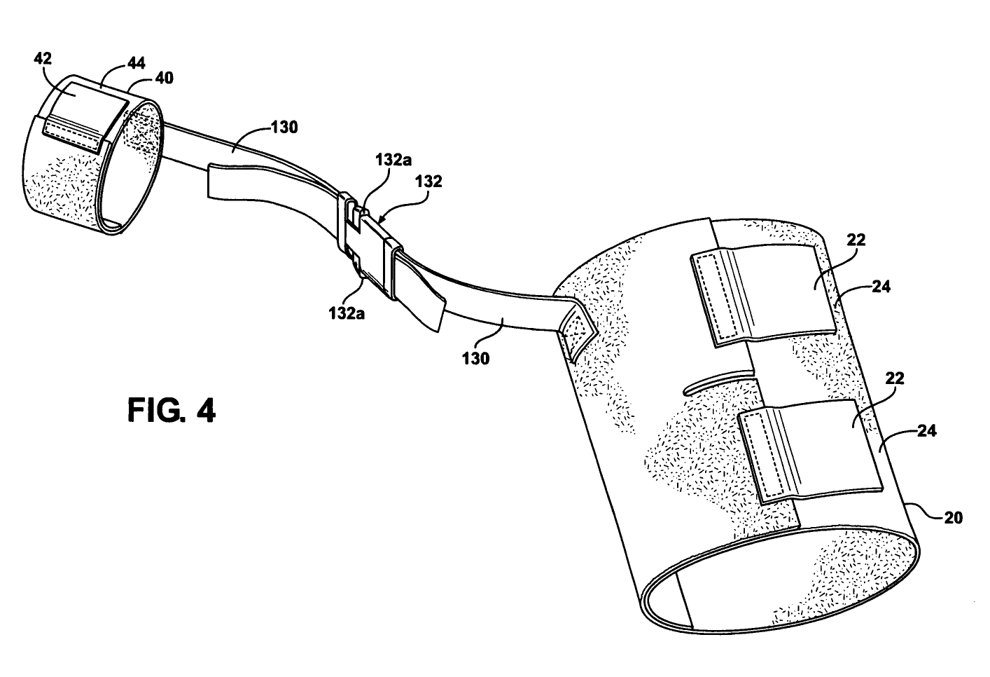

FIG. 4 shows an alternate release mechanism 132, in which a straight (un-looped) strap 130, whose ends are secured to cuffs 20 and 40, is divided by a one-handed release buckle 132 of the well-known Fast-Loc™ type, in which plastic spring detents or fingers 132a on a male half of the fastener yield inwardly and then expand to lock into a female receptacle on the other half of the fastener. The spring fingers can be squeezed with the thumb and fingers of one hand to release the fastener halves, and thus release the wrist cuff 40 from leg cuff 20. Strap(s) 130 can be adjusted in length in known fashion through buckle 132 to adjust the amount of traction-inducing tension.

It will be appreciated that the one-handed release mechanisms 32 and 132 illustrated in FIGS. 3 and 4 are but two of many types of releasable connection that can be used in strap 30 to allow the cuffed hand to release itself from leg cuff 20. In the preferred form, as illustrated with the release mechanism 32, the release mechanism is located along the length of strap 30 nearer (but still spaced from) the wrist cuff 40 to enable the fingers of the cuffed hand to operate the release mechanism when strap 30 is under tension. It will also be appreciated that the adjustable hook-and-loop and buckle connections in strap 30 shown in FIGS. 1-4 are themselves forms of in-line tensioning or force-adjusting devices, albeit simple mechanical ones.

Whatever the release mechanism used in strap 30, the release function will generally be improved by putting strap 30 under tension (stretching it out) prior to operating the release. This release-enhancing tension is facilitated by the greater distance between the lower-leg mounted cuff 20 and wrist cuff 40, which allow the user to place a greater amount of tension on strap 30 with relative ease; and, by the better anchoring function provided by knee K above cuff 20, which ensures that the leg cuff 20 will not slip upward and reduce tension prior to release.

The ends of strap 30 are preferably secured to the cuffs 20 and 40 on the outside of the leg and on the inside of the wrist when the cuffs are worn, as illustrated in FIG. 1, so that the strap 30 does not tend to cross over the arm or leg when under tension.

Before proceeding to the description of the centrifugal exercise device portion of my present invention, it should be understood that while the preferred form of the traction device includes a one-handed quick release mechanism like that described above, the quick release mechanism is preferred but not necessary, and strap 30 could be a simple elastic strap, with or without length adjustment and release features.

While the illustrated traction device 10 with a wrist-to-lower leg connection is believed to be an invention in its own right, as described in my co-pending U.S. provisional application “Orthopedic Shoulder Restraint and Method” (attorney docket no. JSM-001-P), it is also possible with to use a wrist-to-thigh traction device as disclosed in my U.S. patent application Ser. No. 11/159,942, published as US 2006/0293623 on Dec. 28, 2006 (now abandoned) with the present system and method.

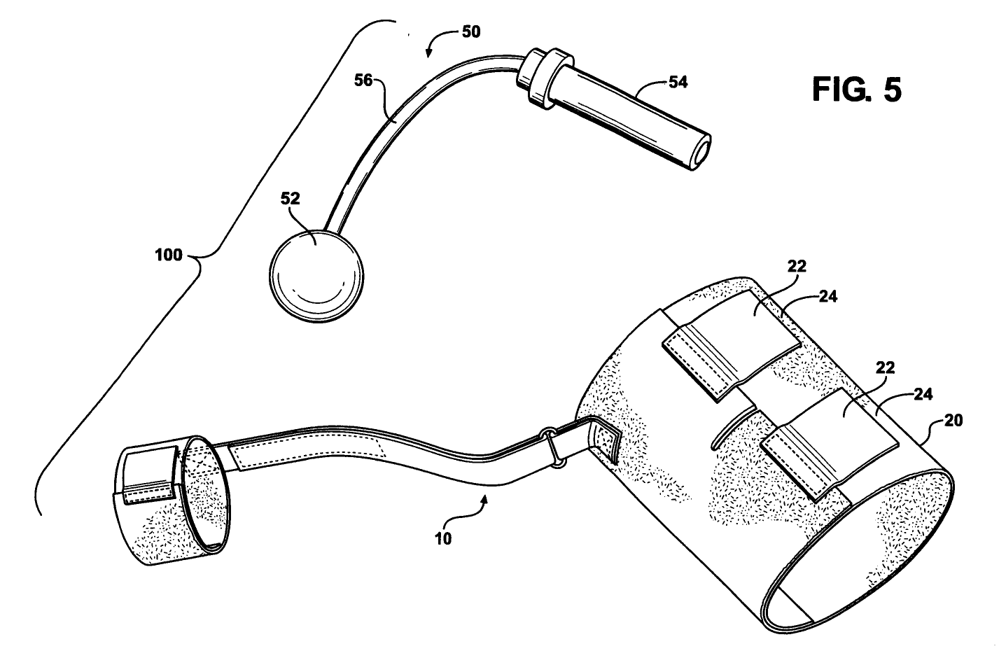

Referring next to FIG. 5, traction device 10 is shown in combination with a centrifugal exercise device 50 to form an impingement-reducing system 100. Device 50 takes the general form of a weighted member 52 rotatably connected to and spaced from a handle 54, in the illustrated embodiment by a short tether 56. Device 50 can be custom built, but the illustrated embodiment is commercially available under the trademark Airope™ and normally used as a “ropeless” or “virtual” jump-rope for people who lack either the physical ability or the space to use a regular jump-rope. While the illustrated centrifugal exercise device 50 shows the weighted member 52 attached to the handle with a tether, an alternate form of suitable device is a ropeless jump-rope such as that shown in U.S. Pat. No. 7,037,243 to Clancy, in which the weighted member rotates in a self-contained track on the end of the handle. Some types of gyroscopic handheld exercise devices of known type, such as the commercially available devices from DynaFlex International under the Dynaflex™ brand name, especially those with gyroscopic force-generating members on the ends of handles, are also believed to be suitable for creating the type of force generated by the ropeless jump-rope type devices when used for the exercises of the present invention. “Spaced” from the handle will accordingly include such tether-less devices wherein the spacing can be the distance between the actual location of the user's hand on the handle and the force-generating rotatable member spaced from the handle, including gyroscopic force-generating members located inside the handle or grip portion as in the Dynaflex “Powerball” device.

Although such ropeless jump-rope devices are known, they are designed and intended to be used as jump-ropes, with a jump-roping motion that would be unsuitable for the inventive method. My invention includes four specific exercises using devices such as 50, which exercises do not mimic jumping rope but instead are targeted for strengthening certain muscle groups to complement the impingement-reduction of the nighttime traction device 10 when done on a regular basis, preferably daily or at least a few times a week. These exercises provide “dynamic stabilization” of the shoulder using the devices like 50, in which the relevant muscles are strengthened with minimal circular motion to strengthen the rotator cuff.

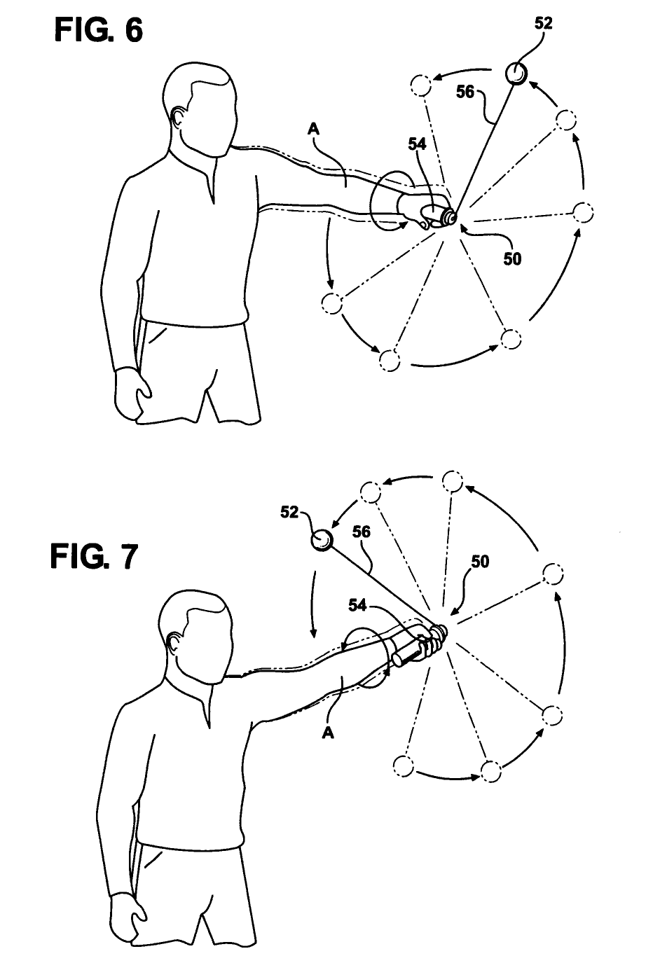

Referring to FIG. 6, the first exercise according to my method is a front vertical plane rotation for specifically strengthening the subscapularis muscles/muscle groups. The person holds centrifugal exercise device 50 by handle 54 in one hand, preferably in a “saber” grip with the index finger and thumb toward the weighted member 52 (toward the upper end of the handle), the handle 54 held straight and essentially horizontal (parallel to the ground) and pointing essentially straight out from the front of the body as pictured, with arm A essentially straight and unbent. In this position, the straight, unbent arm A is rotated through small circles as shown in phantom, clockwise or counterclockwise, such that the weighted member 52 rotates roughly in a circle about the end of handle 54 in an approximately vertical plane.

It will be understood that while terms of position and orientation direction such as horizontal, vertical, straight, circular, front, etc. are used herein to describe the preferred, ideal positions and orientations and motions of the user's body and of the device 50 during the exercises, these terms include natural variations in keeping with a person's ability to approximate them using ordinary visual estimation and physical feel. The range or diameter of the circular arm motion for the exercises described in FIGS. 6-9 can vary, but generally will be the smallest diameter circular motion capable of rotating the weighted member of device 50 sufficiently to generate muscle-strengthening force in the ipsilateral shoulder.

Referring to FIG. 7, a side vertical plane rotation exercise is illustrated for specifically strengthening the subscapularis, infraspinatus, and supraspinatus muscles/muscle groups. The person holds centrifugal exercise device 50 by handle 54 in one hand, preferably in a saber grip with the index finger and thumb toward the weighted member 52, the handle 54 held straight and essentially horizontal (parallel to the ground) and pointing essentially straight out from the side of the body as pictured, with arm A essentially straight and unbent. In this position, the straight, unbent arm A is rotated through small circles, clockwise or counterclockwise, such that the weighted member 52 rotates roughly in a circle about the end of handle 54 in an approximately vertical plane.

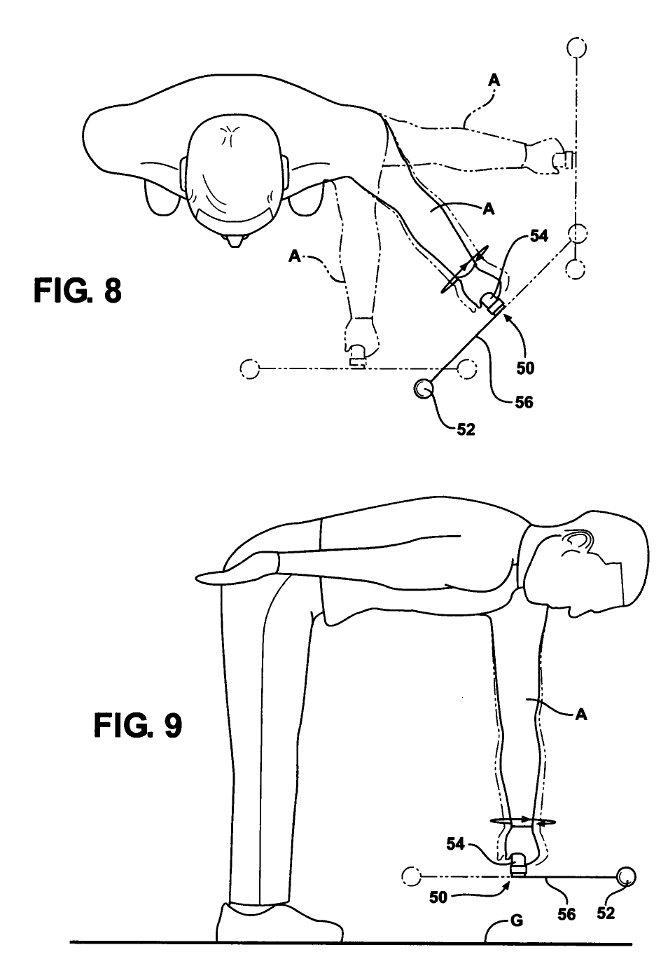

Referring to FIG. 8, a diagonal vertical plane rotation exercise is illustrated for specifically strengthening the subscapularis, infraspinatus, and supraspinatus muscles/muscle groups. The person holds centrifugal exercise device 50 by handle 54 in one hand, preferably in a saber grip with the index finger and thumb toward the weighted member 52, the handle 54 held straight and essentially horizontal (parallel to the ground) and pointing essentially diagonally out from the body as pictured, at approximately 45° (about halfway) between the front and side exercise positions of FIGS. 6 and 7, with arm A essentially straight and unbent. In this position, the straight, unbent arm A is rotated through small circles, clockwise or counterclockwise, such that the weighted member 52 rotates roughly in a circle about the end of handle 54 in an approximately vertical plane.

Referring to FIG. 9, a downward horizontal plane rotation exercise is shown for specifically strengthening the subscapularis, infraspinatus, supraspinatus, and teres minor muscles/muscle groups. The person bends over from the waist, and holds centrifugal exercise device 50 by handle 54 in one hand, again preferably in a saber grip with the index finger and thumb toward the weighted member 52, the handle 54 held straight and essentially vertical (pointing down essentially perpendicular to the ground G) and essentially straight out from the front of the bent-over body as pictured, with arm A essentially straight and unbent. In this position, the straight, unbent arm A is rotated through small circles, clockwise or counterclockwise, such that the weighted member 52 rotates roughly in a circle about the end of handle 54 in an approximately horizontal plane.

### Description of Operation

In operation, the traction device and the centrifugal exercise device are used in two separate but complementary phases. The first phase occurs at night during sleep (or during other prone or supine periods) when traction device 10 is put in place on the body by attaching cuff 20 to the leg (preferably the lower leg) in a range between the ankle and the knee, preferably just below the knee; by attaching cuff 40 to the wrist in a range between the elbow and the hand, preferably right at the wrist; and, if an adjustment mechanism is provided, by adjusting the length of strap 30 for optimum impingement-reducing tension on the ipsilateral shoulder. If the user wants to disconnect the wrist cuff 40 from leg cuff 20 while leaving the cuffs attached to their respective wrist and leg, he can use either the ipsilateral (cuffed) hand to break the strap connection between the cuffs using mechanism 32 or 132, or he can use the opposite hand (if available) to operate the release mechanism with equal ease. Of course, using one or both hands, the user can unwrap or otherwise remove cuffs 20 and 40 from the lower leg and wrist by undoing fasteners 22 and 42. The “nighttime” phase is thus the wearing of traction device 10 for a reasonably extended period of time, usually measured in hours, whatever the actual time of day.

The second phase occurs when the user is awake and active, and involves the use of centrifugal exercise device 50 for one or more (preferably all) of the four exercises described above in reference to FIGS. 6-9. The exercises are preferably performed on a daily or at least several-times-a-week basis for a period corresponding to the period during which traction device 10 is worn at night, for example several weeks or months.

The combination of the impingement-reduction while sleeping using traction device 10, and the strengthening of the impingement-reducing muscles of the shoulder area using centrifugal exercise device 50 to perform the exercises described above, has been found to be a significant improvement over previous devices and methods in terms of rotator cuff tendonitis relief.

It will be understood that while the disclosed embodiments illustrate presently preferred forms of the invention(s), they are intended to be explanatory rather than limiting. Reasonable variations and modifications of the examples disclosed in the foregoing description and drawings are possible without departing from the scope of the claimed subject matter. The scope of the invention(s) is defined by the following claims.

What is claimed:

1. An orthopedic kit to prevent impingement in a user's shoulder, comprising:
a traction device for preventing abduction of the user's ipsilateral arm while the user is in a sleeping position, comprising a wrist cuff adapted to be removably secured to the user's ipsilateral wrist or lower arm, a leg cuff adapted to be removably secured to the user's ipsilateral leg; and a strap member connected between the wrist cuff and the leg cuff and of a length to apply impingement-reducing traction on the user's ipsilateral arm and thus on the user's shoulder while in a sleeping position and with the user's ipsilateral arm alongside the user's body; and,
a centrifugal exercise device for use when awake and upright for strengthening the rotator cuff impingement-preventing muscle group of the shoulder of the user, comprising a handle and a weighted member rotatably connected to the handle so that a user gripping the handle with the ipsilateral arm unbent can rotate the weighted member in an essentially circular path that defines a plane substantially perpendicular to the unbent ipsilateral arm of the user.

2. The orthopedic kit of claim 1, wherein the leg cuff is adapted to be attached to a thigh portion of the user's ipsilateral leg.

3. The orthopedic kit of claim 1, wherein the leg cuff is adapted to be attached to a below-the-knee portion of the user's ipsilateral leg.

4. The orthopedic kit of claim 1, wherein the strap is elastic.

5. The orthopedic kit of claim 1, wherein the strap member includes a quick release mechanism for breaking the strap connection between the wrist cuff and the leg cuff while leaving the wrist and leg cuffs attached to their respective wrist and leg.

6. The orthopedic kit of claim 1 and further comprising multiple centrifugal exercise devises, each with a weighted member of different magnitude.

7. A method for using the orthopedic kit of claim 1 to strengthen a user's shoulder, comprising:
preventing impingement during a nighttime phase using the traction device for preventing abduction of the user's ipsilateral arm while the user is in a sleeping position by:
removably securing the wrist cuff to the user's ipsilateral wrist or lower arm;
removably securing the leg cuff to the user's ipsilateral leg; and
tensioning the strap member between the wrist cuff and the leg cuff to apply impingement-reducing traction on the ipsilateral arm and thus on the ipsilateral shoulder; and
strengthening the user's ipsilateral rotator cuff muscle group of the user's shoulder during an upright phase by using the centrifugal exercise device with the user gripping the handle and rotating the centrifugal exercise device in a circle with the user's ipsilateral arm essentially straight in a manner so that the weighted member defines a plane.

8. The method of claim 7 wherein the rotating act includes rotating the centrifugal exercise device at a speed sufficient for the weighted member to define at least one of a front vertical plane, a side vertical plane, a diagonal vertical plane, and a downward horizontal plane conducted with the user's ipsilateral arm essentially straight.

9. The method of claim 7 wherein the rotating act is repeated at a different time period with a centrifugal exercise with a heavier weight.

10. An orthopedic device to limit abduction of an arm of a user in order to prevent impingement in the user's ipsilateral shoulder, comprising:
a wrist cuff removably secured to the user's ipsilateral lower arm or wrist;
a lower leg cuff removably secured to the user's ipsilateral leg below the knee; and
an elastic strap member connected between the wrist cuff and the lower leg cuff to apply impingement-reducing traction on the ipsilateral arm and shoulder.

11. The device of claim 10, wherein the elastic strap member includes a quick release mechanism for breaking the strap connection between the wrist cuff and the lower leg cuff while leaving the wrist and lower leg cuffs attached to their respective wrist and leg.

12. The device of claim 11, wherein the quick release mechanism is located on the elastic strap member adjacent to but spaced from the wrist cuff.

13. The device of claim 11, wherein the quick release mechanism is operable with the user's ipsilateral hand.

14. A method for strengthening muscles that surround and stabilize a person's shoulder, comprising:
resiliently connecting the person's ipsilateral arm to the person's ipsilateral leg with traction while the person's ipsilateral arm is alongside the person's body;
maintaining the person's ipsilateral arm with the person's ipsilateral arm alongside the person's body during a first time period while the person is in a sleeping position;
subsequent to the first time period, disconnecting the person's ipsilateral arm from traction and from the ipsilateral leg; and
while the person in in an upright position and during a second time period, rotating the person's ipsilateral arm in a straight, unbent condition through small circles while holding with the hand of the ipsilateral arm a handle of a centrifugal device that has a weighted member on the end of a flexible connector between the weighted member and the handle.

15. The method of claim 14 wherein the speed of rotation of the person's ipsilateral arm is sufficient to rotate the weighted member about a circle that defines a plane that is substantially perpendicular to the person's straight, unbent ipsilateral arm.

16. The method of claim 14 wherein the rotation act is carried out with the person's ipsilateral arm positioned forwardly of the person's body and substantially horizontal.

17. The method of claim 14 wherein the rotation act is carried out with the person's ipsilateral arm positioned laterally of the person's body and substantially horizontal.

18. The method of claim 14 wherein the rotation act is carried out with the person's ipsilateral arm positioned forwardly of the person's body and substantially vertical while the person is bent over at the waist.

19. The method of claim 14 wherein the maintaining act is further carried out with the person's ipsilateral arm in traction at least part of the first time period while the person is in a sleeping position.

20. The method of claim 14 wherein the rotation act is repeated during successive time periods with weights of increasing magnitude.
